(19) United States
(12) Patent Application Publication
(10) Pub. No.: US 2018/0362425 A1
(43) Pub. Date: Dec. 20, 2018

(54) PROCESS FOR THE PREPARATION OF GLYCOLS

(71) Applicant: SHELL OIL COMPANY, HOUSTON, TX (US)

(72) Inventors: Evert VAN DER HEIDE, Amsterdam (NL); Timothy Michael NISBET, Amsterdam (NL); Leslie Andrew CHEWTER, Amsterdam (NL); Pieter HUIZENGA, Amsterdam (NL)

(73) Assignee: SHELL OIL COMPANY, HOUSTON, TX (US)

(21) Appl. No.: 16/060,262

(22) PCT Filed: Dec. 7, 2016
(86) PCT No.: PCT/EP2016/080099

§ 371 Date: Jun. 7, 2018

(30) Foreign Application Priority Data
Dec. 9, 2015 (EP) 15198770.8

(51) Int. Cl. C07C 29/132; C07C 31/20; B01J 19/18; B01J 19/24; B01J 21/06; B01J 23/22; B01J 23/26; B01J 23/28; B01J 23/30

(52) U.S. Cl. CPC C07C 29/132; C07C 31/202; C07C 31/205; B01J 19/1812; B01J 19/2415; B01J 2219/00033; B01J 21/066; B01J 23/22; B01J 23/26; B01J 23/28; B01J 23/30; B01J 21/063

(57) ABSTRACT

The invention provides a continuous process for the preparation of ethylene glycol and 1,2-propylene glycol from starting material comprising one or more saccharides, said process comprising providing a feedstock comprising said one or more saccharides in a solvent to a reactor, said reactor having supported therein a heterogeneous hydrogenation catalytic composition and contacting the feedstock therein directly with said heterogeneous hydrogenation catalytic composition and concurrently with a retro-aldol catalyst composition in the presence of hydrogen, wherein said reactor operates in an essentially plug flow manner.

### FIELD OF THE INVENTION

The present invention relates to a process for the preparation of ethylene and propylene glycols from saccharide-containing feedstocks.

### BACKGROUND OF THE INVENTION

Monoethylene glycol (MEG) and monopropylene glycol (MPG) are valuable materials with a multitude of commercial applications, e.g. as heat transfer media, antifreeze, and precursors to polymers, such as PET. Ethylene and propylene glycols are typically made on an industrial scale by hydrolysis of the corresponding alkylene oxides, which are the oxidation products of ethylene and propylene, produced from fossil fuels.

In recent years, increased efforts have focussed on producing chemicals, including glycols, from renewable feedstocks, such as sugar-based materials. The conversion of sugars to glycols can be seen as an efficient use of the starting materials with the oxygen atoms remaining intact in the desired product.

Current methods for the conversion of saccharides to sugars revolve around a hydrogenation/retro-aldol process as described in Angew. Chem. Int. Ed. 2008, 47, 8510-8513.

An important aim in this area is the provision of a process that is high yielding in desirable products, such as ethylene glycol and propylene glycol, and that can be carried out in a commercially viable manner. A preferred methodology for a commercial scale process would be to use continuous flow technology, wherein feed is continuously provided to a reactor and product is continuously removed therefrom. By maintaining the flow of feed and the removal of product at the same levels, the reactor content remains at a more or less constant volume.

Continuous flow processes for the production of glycols from saccharide feedstock have been described in US20110313212, CN102675045, CN102643165, WO2013015955 and CN103731258. A process for the co-production of bio-fuels and glycols is described in WO2012174087.

Typical processes for the conversion of saccharides to glycols require two catalytic species in order to catalyse retro-aldol and hydrogenation reactions. Typically, the hydrogenation catalysts tend to be heterogeneous. However, the retro-aldol catalysts are generally homogeneous in the reaction mixture. Further, the saccharide-containing feedstock is generally in the form of a slurry in a solvent or as a homogeneous saccharide solution. The handling of such a reaction mixture requires careful consideration. Slurry reactors and ebullated bed reactors are taught as preferred options for a saccharides to glycols process in US20110313212 and WO2013015955, in order to deal with such considerations.

It is known that thermal degradation of reaction intermediates can occur in the conversion of saccharides to glycols. Such degradation reduces the overall yield of desired products and increases the complexity of the isolation process of said desired products. It has generally been found that carrying out the reaction with high concentrations of starting materials in a reactor exacerbates this degradation and the formation of by-products.

Typically, the conversion of saccharides to glycols has, therefore, been carried out as a continuous flow process with a high degree of back mixing using a saccharide-containing feedstock comprising a low concentration of saccharide in solvent. Such a process may be carried out, for example, in a continuous flow stirred tank type reactor. In such a system, the concentration of reactants at any one point will be low, preventing any decomposition due to high concentrations. However, in such a process, as some of the back-mixed reaction mixture is continuously removed from the reactor, there will be some material that does not react to completion. This results in a product stream that contains starting material and/or intermediates, reducing the overall yield of the process and requiring separation of the starting material/intermediate from the desired product and disposal or recycling thereof. In addition, back-mixed reactors require a larger reactor volume in comparison to plug-flow reactors to obtain a comparable product capacity.

One solution to overcome this issue was disclosed in WO2015028398, which describes a continuous process for the conversion of saccharides to glycols, in which the reaction takes place first in a stirred reactor, from which a product stream is continuously removed. The product stream is then provided to a finishing reactor, which operates essentially in a plug flow manner. As a high degree of conversion is achieved in the first reactor, the product stream entering the plug flow reactor will inevitably have a low concentration of starting materials and reactive intermediates present therein and thermal degradation is, thus, reduced.

Further optimisation of a process for the conversion of saccharides into glycols is always desirable. It would be preferable to carry out a continuous process to provide glycols from saccharide-containing feedstock in as high a yield as possible. In such a process, it is desirable that substantially full conversion of the starting material and/or intermediates is achieved and formation of by-products is reduced. Minimising the complexity of any reactor system would also be beneficial.

### SUMMARY OF THE INVENTION

Accordingly, the present invention provides a continuous process for the preparation of ethylene glycol and 1,2-propylene glycol from starting material comprising one or more saccharides, said process comprising providing a feedstock comprising said one or more saccharides in a solvent to a reactor, said reactor having supported therein a heterogeneous hydrogenation catalytic composition and contacting the feedstock therein directly with said heterogeneous hydrogenation catalytic composition and concurrently with a retro-aldol catalyst composition in the presence of hydrogen, wherein said reactor operates in an essentially plug flow manner.

### DETAILED DESCRIPTION OF THE INVENTION

The present inventors have surprisingly found that high yields of desirable glycols may be obtained from saccharide starting materials by contacting the starting material in a solvent concurrently with both a hydrogenation catalyst and a retro-aldol catalyst in the presence of hydrogen in a reactor, wherein the hydrogenation catalyst is a heterogeneous catalytic composition supported within the reactor and the reactor operates in an essentially plug flow manner.

The starting material for the subject process comprises at least one saccharide selected from the group consisting of monosaccharides, disaccharides, oligosaccharides and polysaccharides. Examples of polysaccharides include cellulose, hemicelluloses, starch, glycogen, chitin and mixtures thereof.

If the starting material comprises oligosaccharides or polysaccharides, it is preferable that it is subjected to pre-treatment before being fed to the reactor in a form that can be converted in the process of the present invention. Suitable pre-treatment methods are known in the art and one or more may be selected from the group including, but not limited to, sizing, drying, grinding, hot water treatment, steam treatment, hydrolysis, pyrolysis, thermal treatment, chemical treatment, biological treatment. However, after said pre-treatment, the starting material still comprises mainly monomeric and/or oligomeric saccharides. Said saccharides are, preferably, soluble in the reaction solvent.

Preferably, the starting material supplied to the first reactor after any pre-treatment comprises saccharides selected from starch and/or hydrolysed starch. Hydrolysed starch comprises glucose, sucrose, maltose and oligomeric forms of glucose. Said saccharide is suitably present as a solution, a suspension or a slurry in the solvent.

The process of the present invention is carried out in the presence of a solvent. The solvent may be water or a C₁ to C₆ alcohol or polyalcohol (including sugar alcohols) or mixtures thereof. Preferred C₁ to C₆ alcohols include methanol, ethanol, 1-propanol and iso-propanol. Polyalcohols of use include glycols, particularly products of the hydrogenation/retro-aldol reaction, glycerol, erythritol, threitol, sorbitol and mixtures thereof. Preferably, the solvent comprises water.

In the process of the invention, the starting material is reacted with hydrogen in the presence of a heterogeneous hydrogenation catalytic composition, which is supported within the reactor. Said hydrogenation catalytic composition preferably comprises one or more materials selected from transition metals from groups 8, 9 or 10 or compounds thereof, with catalytic hydrogenation capabilities.

More preferably, the hydrogenation catalytic composition comprises one or more metals selected from the list consisting of iron, cobalt, nickel, ruthenium, rhodium, palladium, iridium and platinum. This metal or metals may be present in elemental form or as compounds. It is also suitable that this component is present in chemical combination with one or more other ingredients in the hydrogenation catalytic composition. It is required that the hydrogenation catalytic composition has catalytic hydrogenation capabilities and it is capable of catalysing the hydrogenation of material present in the reactor.

In the process of the invention, the hydrogenation catalytic composition is heterogeneous with respect to the reaction mixture and is supported within the reactor.

In one embodiment, the heterogeneous hydrogenation catalytic composition comprises metals supported on a solid support. In this embodiment, the solid supports may be in the form of a powder or in the form of regular or irregular shapes such as spheres, extrudates, pills, pellets, tablets, monolithic structures. Alternatively, the solid supports may be present as surface coatings, for examples on the surfaces of tubes or heat exchangers. Suitable solid support materials are those known to the skilled person and include, but are not limited to aluminas, silicas, zirconium oxide, magnesium oxide, zinc oxide, titanium oxide, carbon, activated carbon, zeolites, clays, silica alumina and mixtures thereof.

Alternatively, the heterogeneous hydrogenation catalytic composition may be present as Raney material, such as Raney nickel, preferably present in a pelletised form.

The heterogeneous hydrogenation catalytic composition is preferably present in fixed beds. Said fixed beds may also contain inert material in order to ensure a suitable ratio of catalytic composition to feed. Said inert material may comprise, for example, glass spheres or support materials that have not been impregnated with metals. Suitably, the particle size of the inert materials is comparable with that of the catalytic composition.

In another embodiment, the metal may be present unsupported in catalyst beds within the reactor. In this embodiment inert material may also be present within the catalyst bed in order to ensure a suitable ratio of catalytic composition to feed.

The heterogeneous hydrogenation catalytic composition is suitably preloaded into the reactor before the reaction is started.

Within the reactor, the feedstock is also concurrently contacted with a retro-aldol catalyst composition. Said retro-aldol catalyst composition preferably comprises one or more compound, complex or elemental material comprising tungsten, molybdenum, vanadium, niobium, chromium, titanium or zirconium. More preferably the retro-aldol catalyst composition comprises one or more material selected from the list consisting of tungstic acid, molybdic acid, ammonium tungstate, ammonium metatungstate, ammonium paratungstate, tungstate compounds comprising at least one Group I or II element, metatungstate compounds comprising at least one Group I or II element, paratungstate compounds comprising at least one Group I or II element, heteropoly compounds of tungsten, heteropoly compounds of molybdenum, tungsten oxides, molybdenum oxides, vanadium oxides, metavanadates, chromium oxides, chromium sulfate, titanium ethoxide, zirconium acetate, zirconium carbonate, zirconium hydroxide, niobium oxides, niobium ethoxide, and combinations thereof. The metal component is in a form other than a carbide, nitride, or phosphide. Preferably, the retro-aldol catalyst composition comprises one or more compound, complex or elemental material selected from those containing tungsten or molybdenum.

The retro-aldol catalyst composition may be present as a heterogeneous or a homogeneous catalyst composition. In one embodiment, the retro-aldol catalyst composition is heterogeneous and is supported in the reactor along with the heterogeneous hydrogenation catalyst composition. In this embodiment, the retro-aldol catalyst composition may be supported on the same support or a different support to the hydrogenation catalyst composition. In another embodiment, the retro-aldol catalyst composition is homogeneous with respect to the reaction mixture.

Depending on the physical state of the retro-aldol catalyst composition and any components contained therein, they may be preloaded into the reactors or, if they are in liquid form or present as a solution or slurry in a solvent, they may be fed into the reactor as required in a continuous or discontinuous manner during the process of the present invention.

The weight ratio of the retro-aldol catalyst composition (based on the amount of metal in said composition) to sugar feed is suitably in the range of from 1:1 to 1:1000. The weight ratio of the hydrogenation catalyst composition (based on the amount of metal in said composition) to sugar feed is suitably in the range of from 10:1 to 1:100.

The weight ratio of the retro-aldol catalyst composition to the hydrogenation catalyst composition (based on the amount of metal in each composition)is typically in the range of from 1:1000 to 1:1.

The process of the present invention takes place in the presence of hydrogen. Preferably, the process of the present reaction takes place in the absence of air or oxygen. In order to achieve this, it is preferable that the atmosphere in the reactor be evacuated and replaced with an inert gas (e.g. nitrogen or argon) and then hydrogen repeatedly, after loading of any initial reactor contents, before the reaction starts.

The reactor used in the process of the present invention operates in an essentially plug flow manner. In a reactor operating with essentially a plug flow, all of the feed stream moves with the same radially uniform velocity and, therefore, has the same residence time. The concentration of the reactants in the plug flow reactor will change as it progresses through the reactor. Although the reaction mixture preferably essentially completely mixes in radial direction and preferably does essentially not mix in the axial direction (forwards or backwards), in practice some mixing in the axial direction (also referred to as back-mixing) may occur. Suitable reactors operating with essentially plug flow include, but are not limited to, tubular reactors, pipe reactors, falling film reactors, staged reactors, packed bed reactors, trickle bed reactors and shell and tube type heat exchangers. Said reactors may operate in either upflow or downflow operation for the liquid phase, and co-current or counter-current flow for liquid/gas phases.

The plug flow reactor may, for example, be operated in the transition area between laminar and turbulent flow or in the turbulent area, such that a homogenous and uniform reaction profile is created.

A plug flow may for example be created in a tubular reactor. It may also be created in a compartmentalized tubular reactor or in another reactor or series of reactors having multiple compartments being transported forward, where preferably each of these compartments are essentially completely mixed. An example of a compartmentalized tubular reactor operated at plug flow may be a tubular reactor comprising a screw.

Preferably a Péclet number of at least 3, more preferably at least 6, and still more preferably at least 20, most preferably at least 100 is maintained within the plug flow reactor.

The residence time of the liquid phase in the first reactor is suitably at least 1 minute, preferably at least 2 minutes, more preferably at least 5 minutes. Suitably, the residence time in the first reactor is no more than 5 hours, preferably no more than 2 hours, more preferably no more than 1 hour.

The temperature in the reactor is suitably at least 130° C., preferably at least 150° C., more preferably at least 170° C., most preferably at least 190° C. The temperature in the reactor is suitably at most 300° C., preferably at most 280° C., more preferably at most 270° C., even more preferably at most 250° C. Preferably, the reactor is heated to a temperature within these limits before addition of any saccharide starting material and is maintained at such a temperature until all reaction is complete.

The pressure in the reactor is suitably at least 1 MPa, preferably at least 2 MPa, more preferably at least 3 MPa. The pressure in the reactor is suitably at most 25 MPa, preferably at most 15 MPa, more preferably at most 8 MPa. Preferably, the reactor is pressurised to a pressure within these limits by addition of hydrogen before addition of any saccharide starting material and is maintained at such a pressure until all reaction is complete through on-going addition of hydrogen.

After the feedstock been contacted with the heterogeneous hydrogenation catalytic composition and a retro-aldol catalyst composition in the presence of hydrogen, in the reactor, according to the process of the present invention, suitably at least 98 wt %, preferably at least 99 wt %, more preferably at least 99.5 wt % of the starting material has reacted to completion. Reacting to completion means that the starting material and any unsaturated components such as ketones and aldehydes, more specifically hydroxyl-ketones and hydroxyl-aldehydes are no longer present in the reaction mixture.

The present invention is further illustrated in the following Examples.

### EXAMPLES

A kinetic model was developed in order to accurately model the combined retro-aldol and hydrogenation reactions of saccharides. These reactions result in a range of products, including ethylene glycol, propylene glycol, 1,2-butanediol, 1,2-hexanediol, glycerol, erythritol, threitol and sorbitol. Known intermediates include glycolaldehyde, hydroxyacetone and 1-hydroxy-2-butanone.

Reaction rates are not available in the literature for each individual reaction. Therefore, reaction rates were averaged on the basis of literature available (Zhang, J., Hou, B., Wang, A., Li, Z., Wang, H., Zhang, T., AIChE Journal (2014) 60 (11) 3804-3813; Zhang, J., Hou, B., Wang, A., Li, Z., Wang, H., Zhang, T., AIChE Journal (2015) 61 (1) 224-238), with the exception of hydrogenation of glycolaldehyde, which was assumed to be five times faster than the average rate of hydrogenation of all other ketone and aldehyde intermediates (referring to Mahfud, F. H., Ghijsen, F., Heeres, H. J., Journal of Molecular Catalysis A: Chemical (2007) 264 (1-2) 227-236).

The kinetic model was set up in Microsoft Excel 2010 and the respective pre-exponential factors and activation energies were slightly adjusted to enable fitting of experimental data of glucose conversion during time at various temperatures. As a final check, concentrations and conditions provided in literature (Zhao, G., Zheng, M., Zhang, J., Wang, A., Zhang, T., Ind. Eng. Chem. Res. (2013) 52 (28) 9566-9572) were used as input, resulting in model predictions in reasonable accordance with the product yields reported.

The kinetic model was then used to predict the outcome of a process according to the present invention. A feed stream comprising 10% w glucose in water and varying concentrations of tungsten as a retro-aldol catalyst were fed to an ideal plug flow reactor, assumed to be charged with varying amounts of hydrogenation catalyst (Table 1). The model unit for hydrogenation represents hydrogenation activity and combines catalyst concentration (expressed in gram/liter) and hydrogen pressure. The table clearly indicates that high MEG and MPG yields are predicted, increasing with increasing catalyst concentrations.

A MEG yield of 67.98% w yield was predicted after increasing the glucose feed concentration from 10% w to 40% w (Table 1).

Yields are given in weight percent and calculated as weight of product divided by the weight of saccharide feed and multiplied by 100.

TABLE 1
product yields

| | | | | | | | |
|---|---|---|---|---|---|---|---|
| Catalysts | | | | 1,2- | | erythritol/ | |
| W | hydrogenation | MEG | MPG | butanediol | glycerol | threitol | sorbitol |
| (ppmw) | (model unit) | (% w) | (% w) | (% w) | (% w) | (% w) | (% w) |

10 wt % glucose feed

| | | | | | | | |
|---|---|---|---|---|---|---|---|
| 100 | 6 | 54.745 | 6.422 | 3.254 | 2.606 | 6.886 | 14.378 |
| 333 | 6 | 60.394 | 9.369 | 5.821 | 1.481 | 2.548 | 4.790 |
| 1000 | 20 | 67.010 | 9.241 | 5.684 | 1.587 | 2.801 | 5.295 |
| 3333 | 20 | 70.010 | 10.857 | 7.231 | 0.621 | 0.906 | 1.649 |
| 10000 | 60 | 73.232 | 10.857 | 7.231 | 0.621 | 0.906 | 1.649 |

40 wt % glucose feed

| | | | | | | | |
|---|---|---|---|---|---|---|---|
| 10000 | 60 | 67.980 | 10.857 | 7.231 | 0.621 | 0.906 | 1.649 |

1. A continuous process for the preparation of ethylene glycol and 1,2-propylene glycol from starting material comprising one or more saccharides, said process comprising providing a feedstock comprising said one or more saccharides in a solvent to a reactor, said reactor having supported therein a heterogeneous hydrogenation catalytic composition and contacting the feedstock therein directly with said heterogeneous hydrogenation catalytic composition and concurrently with a retro-aldol catalyst composition in the presence of hydrogen, wherein said reactor operates in an essentially plug flow manner.

2. The process as claimed in claim 1, wherein the starting material supplied to the first reactor comprises starch and/or hydrolysed starch.

3. The process as claimed in claim 1, wherein the solvent is water or a C₁ to C₆ alcohol or polyalcohol including sugar alcohols or mixtures thereof.

4. The process as claimed in claim 1, wherein the heterogeneous hydrogenation catalytic composition comprises one or more materials selected from transition metals from groups 8, 9 or 10 or compounds thereof, with catalytic hydrogenation capabilities.

5. The process as claimed in claim 1, wherein the retro-aldol catalyst composition comprises one or more compound, complex or elemental material comprising tungsten, molybdenum, vanadium, niobium, chromium, titanium or zirconium.

6. The process as claimed in claim 1, wherein the retro-aldol catalyst composition is homogeneous with respect to the reaction mixture.

7. The process as claimed in claim 1, wherein the weight ratio of the retro-aldol catalyst composition, based on the amount of metal in said composition, to sugar feed is suitably in the range of from 1:1 to 1:1000.

8. The process as claimed in claim 1, wherein the weight ratio of the hydrogenation catalyst composition, based on the amount of metal in said composition, to sugar feed is suitably in the range of from 10:1 to 1:100.

9. The process as claimed in claim 1, wherein the first reactor is selected from the group consisting of tubular reactors, pipe reactors, falling film reactors, staged reactors, packed bed reactors, trickle bed reactors and shell and tube type heat exchangers.

10. The process as claimed in claim 1, wherein the temperature in the reactor is at least 170° C. and at most 250° C. and the pressure in the reactor is suitably at least 1 MPa and at most 8 MPa.
